(19) United States
(12) Patent Application Publication
(10) Pub. No.: US 2007/0141095 A1
(43) Pub. Date: Jun. 21, 2007

(54) AQUEOUS DISPERSION OF COLLOIDAL PARTICLES OF MINERAL FILLER AND FIBRES

(75) Inventor: Jean-Thierry SIMONNET, Cachan (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/609,511

(22) Filed: Dec. 12, 2006

Related U.S. Application Data
(60) Provisional application No. 60/752,008, filed on Dec. 21, 2005.

(30) Foreign Application Priority Data
Dec. 15, 2005 (FR) 05 53894

(51) Int. Cl. A61Q 19/08; A61K 8/02

(52) U.S. Cl. 424/401; 424/70.27; 514/937; 514/938

(57) ABSTRACT

The invention relates to a composition containing an aqueous dispersion of colloidal particles of at least one mineral filler, and fibres. The invention also relates to a process for treating the skin, especially wrinkled skin, via the application of the described composition to the skin.

### REFERENCE TO PRIOR APPLICATIONS

This application claims priority to U.S. provisional application 60/752,008 filed Dec. 21, 2005, and to French patent application 0553894 filed Dec. 15, 2005, both incorporated herein by reference.

### FIELD OF THE INVENTION

The present invention relates to an aqueous dispersion of colloidal particles of at least one mineral filler, in particular silica, and organic fibres, and to compositions comprising a tensioning agent using this dispersion. The invention further relates to a cosmetic skincare process.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In the course of the ageing process, impairment in the skin structure and functions appears. The main clinical signs observed are the appearance of wrinkles and fine lines associated with slackening of the skin. A person skilled in the art knows that such slackening can be corrected immediately by applying a tensioning agent to the skin.

The use of many tensioning agents for treating wrinkles is known to those skilled in the art at the present time. The ones that particularly come to mind are aqueous dispersions of colloidal or non-colloidal particles of a mineral filler, in particular silica, for smoothing out wrinkles via a tensioning effect, which are described especially in documents U.S. Pat. No. 4,819,825, U.S. Pat. No. 4,777,041, US2002/0098220, FR-2 823 113, FR-2 659 551 and WO 02/15873.

Unfortunately, compositions containing such tensioning agents, although having a very satisfactory tensioning effect, have the major drawback of having a limited effect over time.

### BRIEF DESCRIPTION OF THE DRAWING

FIG. 1 shows an example of a curve of force as a function of displacement used to determine the breaking point of the material.

### SUMMARY OF THE INVENTION

Consequently, there is a great need for compositions that simultaneously show excellent efficacy and for which the tensioning effect is long-lasting. The inventor has now discovered that the inclusion of fibres into compositions comprising a synthetic polymeric tensioning agent makes it possible to improve the mechanical properties of the polymeric tensioning film to allow it especially to mimic facial expressions without cracking, and thus to improve its remanence (e.g., long-lasting effects).

### DETAILED DESCRIPTION OF THE PREFERRED EMBODIMENTS

One subject of the present invention is thus a composition, preferably a cosmetic composition, comprising, preferably in a physiologically acceptable medium, at least:
one aqueous dispersion of colloidal particles of at least one mineral filler, the particles having a number-average diameter of between 0.1 and 100 nm, and
at least one type of organic fibres.

Another subject of the invention is a process for caring for the skin, in particular wrinkled skin, comprising the topical application to the skin of a composition comprising, preferably in a physiologically acceptable medium, at least:
one aqueous dispersion of colloidal particles of at least one mineral filler, the particles having a number-average diameter of between 0.1 and 100 nm, and
at least one type of organic fibres.

The use of fibres in cosmetics is described in many documents. Particular mention may be made, inter alia, of documents EP 1 090 626, EP 1 090 627, EP 1 092 424, EP 1 243 251 and EP 1 262 168. However, in none of these documents is a dispersion of colloidal particles of at least one mineral filler combined with organic fibres, to correct the signs of ageing.

The composition and the process according to the invention are in particular intended for smoothing out human facial and/or bodily skin and/or for reducing or effacing the signs of ageing of the skin, in particular for reducing or effacing skin wrinkles and/or fine lines.

The colloidal particles of at least one mineral filler are advantageously present in an effective amount in the composition used according to the invention. In this case, the term “effective amount” means the amount at least equal to the amount required to give the composition a tensioning effect that is visible to the naked eye. Similarly, the fibres are preferably present in an effective amount in the composition used according to the invention. In this case, the term “effective amount” means an amount at least equal to the amount required for this tensioning effect to be substantially remanent, i.e. still visible at least one hour, preferably at least two hours and better still at least five hours, after application of the composition.

The tensioning and remanent effects are especially measurable by scoring photographs obtained at different times after application of a product to an area bearing wrinkles. The “fringe projection” technique may also be used, which allows fine quantification of the modifications of the microrelief induced by a product.

The term “physiologically acceptable medium” means a medium free of toxicity, which is compatible with the skin and optionally with its integuments, mucous membranes and semi-mucous membranes.

In the context of the present invention, the expressions “between . . . and . . . ”, “ranging between . . . and . . . ” and “ranging from . . . to . . . ” mean that the limits are also included.

As stated previously, the compositions according to the invention comprise at least one dispersion of colloidal particles of at least one mineral filler. For the purposes of the present invention, the term “colloidal particles” means particles with a number-average diameter of between 0.1 and 100 nm, preferably between 3 and 30 nm and better still between 10 and 15 nm, including 0.5, 0, 5, 10, 15, 20, 25, 30, 35, 40, 45, 50, 55, 60, 65, 70, 75, 80, 85, 90 and 95 nm, including all values and subranges between stated values. These particles tend to conserve the above particularly mentioned diameters in the composition containing them, without aggregating, and therefore preferably do not have thickening properties, in the sense that at a concentration of greater than or equal to 15% by weight in water, the colloidal particles according to the invention have a viscosity of less than 0.05 Pa·s for a shear rate equal to 10⁻¹ s, the viscosity being measured at 25° C. using a Haake RheoStress RS150 rheometer in cone-plate configuration, the cone having a diameter of 60 mm and an angle of 2°.

The invention dispersion of colloidal particles may be prepared according to the “sol-gel” process that is well known to those skilled in the art, from salts or alkoxides of the corresponding metals dissolved in a solvent, such as an alcohol. A hydrolysis reaction is then performed to form an amorphous precipitate. The mixture is then dispersed in water with an acid or a base at the desired pH, which leads to peptization of the precipitate and crystallization. A crystalline oxide dispersed in water is thus formed.

Aqueous colloidal suspensions may be prepared, for example, according to the processes described in J. Colloïd Interface Sci., 26, p. 62-69, 1968 for SiO₂, Appli. Opt. 26, 4688, 1987 for TiO₂, Inorg. Chem., 3, 146, 1964 for ZrO₂, Appl. Opt., 27, 3356, 1988 for CaF₂ and MgF₂. These suspensions are prepared using ionic precursors usually chosen from chlorides, oxychlorides, perchlorates, nitrates, oxynitrates or acetates, or molecular precursors preferably chosen from alkoxides, of molar formula M(OR)ₙ (M representing a metal, OR an alkoxy radical of 1 to 6 carbon atoms and n representing the valency of the metal). In the methods described previously, the precursor is hydrolysed or fluorinated and then polymerized until a finished product, which is insoluble in the chosen solvent, nucleated and referred to as a colloidal suspension is obtained. In the case of alkoxides, the hydrolysis must be vigorously controlled, given the highly hydrophilic nature of these organometallic derivatives.

Another process for preparing such products is described by J. Livage et al. in “Sol-gel Synthesis of Metal Oxyde Clusters and Colloids” (Mat. Res. Soc. Synt. Proc., Volume 272, pages 3 to 14).

The mineral filler used in the composition according to the invention may preferably be chosen from: silica, cerium oxide, zirconium oxide, alumina, silicon carbonate, barium sulfate, calcium sulfate, zinc oxide, titanium dioxide and platinum.

As examples of aqueous dispersions of colloidal silicas that may be used according to the invention, particular mention may be made of those sold by the company Catalysts & Chemicals under the trade names Cosmo S-40® and Cosmo S-50®.

As a variant, the colloidal particles may consist of a mixed oxide, in particular of a silica-alumina composite filler.

For the purposes of the present invention, the term “particles of silica-alumina composite filler” means particles consisting of silicon oxide and whose surface has been chemically modified so as to replace at least some of the silicon atoms with aluminium atoms, forming at most one monomolecular layer of aluminium. The proportion of their surface area covered with aluminium is generally between 1% and 100%, preferably between 1% and 10% and better still between 4% and 6%. These particles generally have a zeta potential of less than −20 mV and more preferentially less than −25 mV, at pH 7 and at 25° C., as measured using a Delsa 440SX machine from Coulter Scientific Instrument. They may especially be prepared, as described in U.S. Pat. No. 2,892,797, by mixing a silica sol with a sodium aluminate. They are moreover commercially available from the company Grace under the trade references Ludox AMX 6021®, Ludox HSA® and Ludox TMA®.

The amount of colloidal particles present in the composition may vary within a wide range as a function of the desired effect. By way of example, these particles may be present in an active material content ranging from 0.01% to 15% by weight and preferably ranging from 1% to 10% by weight relative to the total weight of the composition.

The aqueous dispersion of colloidal particles of mineral filler is combined in the compositions according to the invention with organic fibres. The shape factor, the yarn count and the morphology of the fibres are the three important factors for defining a fibre.

The fibres that may be used in the compositions according to the invention may be short or long, and individual or organized, for example braided. They are preferably generally of cylindrical shape, in contrast with platelets of parallelepipedal shape and with spherical particles of spherical shape. They may have any morphology and may especially have a circular or polygonal (especially square, triangular, hexagonal or octagonal) cross section according to the specific intended use.

In particular, their extremities may preferably be blunted and/or polished to prevent injury. In particular, the fibres may have a length (L) ranging from 1 μm to 10 mm, preferably from 0.1 mm to 5 mm and better still from 0.1 mm to 1.5 mm. Their cross section may be within a circle of diameter (D) ranging from 1 nm to 100 μm, preferably ranging from 1 μm to 50 μm and better still from 5 μm to 40 μm. Preferably, the fibres used according to the present invention have a shape factor, i.e. a ratio L/D (length/diameter) ranging from 3.5 to 2500, better still from 5 to 500 and even better still from 5 to 150.

The yarn count of the fibres is often given in denier or decitex. The denier is the weight in grams per 9 km of yarn. Preferably, the fibres used in the composition according to the invention have a yarn count ranging from 0.15 to 30 denier and better still from 0.18 to 18 denier.

The fibres that may be used in the composition of the invention include hydrophilic and hydrophobic organic fibres, of synthetic or natural origin.

The organic fibres may be those used in the manufacture of textiles, and especially silk fibres, cotton fibres, wool fibres, flax fibres, cellulose fibres extracted especially from wood, plants or algae, polyamide fibres (Nylon®), modified cellulose fibres (rayon, viscose or acetate, especially rayon acetate), poly-p-phenyleneterephthalamide fibres, especially Kevlar®, acrylic fibres, especially polymethyl methacrylate or poly(2-hydroxyethyl methacrylate) fibres, polyolefin fibres and especially polyethylene or polypropylene fibres, aramid fibres, polytetrafluoroethylene (especially Teflon®) fibres, insoluble collagen fibres, polyester fibres, polyvinyl chloride or polyvinylidene chloride fibres, polyvinyl alcohol fibres, polyacrylonitrile fibres, chitosan fibres, polyurethane fibres, polyethylene phthalate fibres, and fibres formed from a mixture of polymers such as those particularly mentioned above, for instance polyamide/polyester fibres. Examples of polyurethane fibres that may be particularly mentioned include fibres made of poly(urethane-urea) polymer, belonging to the class of elastanes, and especially those sold under the name Lycra by the company DuPont.

The organic fibres that may be used in the composition according to the invention are preferably chosen from polyamide fibres, poly-p-phenyleneterephthalamide fibres and cotton fibres, and mixtures thereof.

It is also possible to use resorbable synthetic organic fibres used in surgery, for instance fibres prepared from glycolic acid and caprolactone (Monocryl from the company Johnson & Johnson); resorbable synthetic fibres of the type such as the copolymer of lactic acid and glycolic acid (Vicryl from the company Johnson & Johnson); terephthalic polyester fibres (Ethibond from the company Johnson & Johnson). Mixtures of the fibres particularly mentioned above may also be used.

Moreover, the fibres may or may not be surface-treated, and may be coated or uncoated. They may especially be coated and/or functionalized fibres. As coated fibres that may be used in the invention, particularly mention may be made of polyamide fibres coated with copper sulfide to give an antistatic effect (for example R-Stat from the company Rhodia) or another polymer having a particular organization of the fibres (specific surface treatment) or a surface treatment that induces colour/hologram effects (for example Lurex fibre from the company Sildorex).

The fibres may also be functionalized, i.e. modified, especially surface-treated, so as to have a specific function or modified properties. This functionalization of the fibres may be performed either on the fibres or in the fibres, and by any method allowing a compound to be attached to the fibres or to be trapped in the cavities formed by the geometry of the fibres. Examples of methods that may be particularly mentioned include the coating of the fibres with an active agent; the fixing of particles containing an active agent, such as nanocapsules or nanospheres, onto the fibres; adsorption into the fibres; fixing via chemical reaction. It is also possible to use fibres that have particular functionalities, for example anti-UV fibres by modification with chemical or physical sunscreens; bactericidal or antiseptic fibres by modification with preserving agents or antibacterial agents; coloured fibres by modification with colouring molecules; keratolytic or desquamating fibres by modification with keratolytic or desquamating agents; moisturizing fibres by modification with moisturizers or water-retaining polymers; fragranced fibres by modification with a fragrance; analgesic or calmative fibres by modification with an anti-inflammatory agent or a calmative; antiperspirant fibres by modification with an antiperspirant agent.

It is in particular possible to use the polyamide fibres sold by Etablissements P. Bonte under the name Polyamide 0.9 dtex 0.3 mm, with a mean diameter of from 15 to 20 μm, a yarn count of about 0.9 dtex (0.81 denier) and a length ranging from 0.3 mm to 1.5 mm. It is also possible to use poly-p-phenyleneterephthalamide fibres with a mean diameter of 12 μm and a length of about 1.5 mm, for instance those sold under the name Kevlar Floc by the company DuPont Fibres. These polyamide fibres are preferably introduced into an oily medium or via a dry route into a powder. It is also possible to use cotton fibres with a mean diameter of 20 μm, a length of 0.3 mm and a shape factor of 15, such as those sold by the company Filature de Lomme, by the Institut Textile de France, by the company Textiles des Dunes or by the company Velifil.

Depending on their properties, the fibres used according to the present invention may be introduced into an aqueous medium, an oily medium and/or into a powder.

The organic fibres may be present in the composition according to the invention in any amount, including an amount ranging from 0.1% to 50% by weight, preferably from 0.5% to 30% by weight, better still from 1% to 20% by weight and even better still from 0.1% to 15% (and especially from 1% to 15% by weight and better still from 2% to 15% by weight), or even from 0.1% to 10% by weight (and especially from 1% to 10% by weight and better still from 2% to 10% by weight), and preferentially ranging from 0.1% to 8% by weight (and especially from 1% to 8% by weight and better still from 2% to 8% by weight), and more preferentially ranging from 0.1% to 5% by weight (and especially from 1% to 5% by weight and better still from 2% to 5% by weight), relative to the total weight of the composition.

The compositions according to the invention preferably comprise, in addition to the organic fibres and the aqueous dispersion of colloidal particles of at least one mineral filler, a physiologically acceptable medium.

The composition of the invention may be in any form, including any galenical form normally used for topical application to the skin, especially in the form of an aqueous, aqueous-alcoholic or oily solution, an aqueous or oily gel, a liquid, pasty or solid anhydrous product, a dispersion of oil in an aqueous phase in the presence of spherules, these spherules possibly being polymeric nanoparticies such as nanospheres or nanocapsules or, better still, lipid vesicles of ionic and/or nonionic type, or a direct emulsion (O/W), inverse emulsion (W/O) or multiple emulsion (O/W/O and W/O/W). The composition used according to the invention is preferably in the form of an aqueous gel or an oil-in-water emulsion.

For example, when it is applied to the skin, this composition may be more or less fluid and may have the appearance of a white or coloured cream, a milk, a lotion, a serum, a paste or a mousse. It may optionally be applied to the skin in aerosol form. It may also be in solid form, for example in the form of a stick or a compact product. It may be used as a care product and/or as a skin makeup product. For example, it may be used as a foundation.

As particularly mentioned previously, the compositions used according to the invention may comprise an aqueous phase.

This aqueous phase may mainly contain water. It may also comprise a mixture of water and of water-miscible organic solvent (miscibility in water of greater than 50% by weight at 25° C.).

This aqueous phase may typically be present in a content of greater than or equal to 10%, preferably 30%, even more preferably 50%, or even 70% by weight relative to the total weight of the composition.

In a known manner, the composition of the invention may also contain adjuvants such as those that are common in cosmetics, such as hydrophilic or lipophilic gelling agents, hydrophilic or lipophilic active agents, preserving agents, antioxidants, solvents, fragrances, fillers, screening agents, pigments, odour absorbers and dyestuffs. The amounts of these various adjuvants are those conventionally used in the field under consideration, for example from 0.01% to 20% of the total weight of the composition. Depending on their nature, these adjuvants may be introduced into the fatty phase, into the aqueous phase, into lipid vesicles and/or into nanoparticies. These adjuvants and the concentrations thereof should be such that they do not modify the desired property of the tensioning agent.

When the composition of the invention is an emulsion, the proportion of the fatty phase may range from 5% to 80% by weight and preferably from 5% to 50% by weight relative to the total weight of the composition. The fatty substances, emulsifiers and co-emulsifiers used in the composition in emulsion form are chosen from those conventionally used in the field under consideration. The emulsifier and the co-emulsifier are preferably present in the composition in a proportion ranging from 0.3% to 30% by weight and preferably from 0.5% to 20% by weight relative to the total weight of the composition.

As fatty substances that may be used in the invention, particularly mention may be made of oils and especially mineral oils (liquid petroleum jelly), oils of plant origin (avocado oil or soybean oil), oils of animal origin (lanoline), synthetic oils (perhydrosqualene), silicone oils (cyclomethicone) and fluoro oils (perfluoropolyethers). Fatty alcohols (cetyl alcohol), fatty acids, waxes and gums, and in particular silicone gums, may also be used as fatty substances

As emulsifiers and co-emulsifiers that may be used in the invention, examples that may be particularly mentioned include fatty acid esters of polyethylene glycol, such as PEG-50 stearate and PEG-40 stearate, and fatty acid esters of polyols, such as glyceryl stearate and sorbitan tristearate.

Hydrophilic gelling agents that may be particularly mentioned in particular include carboxyvinyl polymers (carbomer), acrylic copolymers such as acrylate/alkylacrylate copolymers, polyacrylamides, polysaccharides, natural gums and clays, and lipophilic gelling agents that may be particularly mentioned include modified clays, for instance bentones, metal salts of fatty acids, hydrophobic silica and polyethylenes.

Especially when it is used to efface the signs of ageing of the skin, the composition used according to the invention may preferably comprise, as active agents, at least one compound chosen from: desquamating agents and/or moisturizers; depigmenting agents; anti-glycation agents; agents for stimulating the synthesis of dermal or epidermal macromolecules and/or for preventing their degradation; agents for stimulating fibroblast and/or keratinocyte proliferation or for stimulating keratinocyte differentiation; muscle relaxants; antipollution agents and/or free-radical scavengers; and mixtures thereof.

Examples of such active agents are: retinol and its derivatives such as retinyl palmitate; ascorbic acid and its derivatives such as magnesium ascorbyl phosphate and ascorbyl glucoside; tocopherol and its derivatives such as tocopheryl acetate; nicotinic acid and its precursors such as nicotinamide; ubiquinone; glutathione and its precursors such as L-2-oxothiazolidine-4-carboxylic acid; plant extracts, especially extracts of samphire and of olive leaves, and also plant proteins and hydrolysates thereof such as rice or soybean protein hydrolysates; algal extracts and in particular extracts of laminaria; bacterial extracts; sapogenins such as diosgenin and extracts of Dioscorea plants, in particular wild yam, containing them; α-hydroxy acids; β-hydroxy acids, such as salicylic acid and 5-n-octanoylsalicylic acid; oligopeptides and pseudodipeptides and acylated derivatives thereof, in particular {2-[acetyl(3-trifluoromethylphenyl)amino]-3-methylbutyrylamino}acetic acid and the lipopeptides sold by the company Sederma under the trade names Matrixyl 500 and Matrixyl 3000; lycopene; manganese and magnesium salts, in particular gluconates; and mixtures thereof.

In addition, the tensioning agents used according to the invention may also be combined with other compounds known to those skilled in the art as tensioning agents, especially plant proteins, polysaccharides of plant origin optionally in the form of microgels, starches and mixed silicates.

When it is intended for smoothing out bodily skin, in particular as a slimming and/or firming composition, the composition used according to the invention may comprise, as a variant, one or more draining, lipolytic, de-infiltrating, slimming, firming, anti-glycating and/or vasoprotective compounds.

Examples of such compounds may be chosen from: phosphodiesterase inhibitors such as caffeine and theobromine; monomethylsilanetriol mannuronate; extracts of tea, coffee, guarana, maté, or cola (Cola nitida); extracts of Ginkgo biloba; extracts of escin; extracts of Dioscorea plants containing diosgenin; algal extracts and in particular extracts of Laminaria digitata; and mixtures thereof.

In the event of incompatibility, the active agents indicated above may be incorporated into spherules, especially ionic or nonionic vesicles and/or nanoparticles (nanocapsules and/or nanospheres), so as to isolate them from each other in the composition.

### EXAMPLES

### Example 1

Anti-wrinkle Compositions

The examples below of compositions are given as non-limiting illustrations. In these examples, the compounds bear their INCI name.

Examples 1A and 1B below were prepared according to the procedure below.

Phase B (with the exception of the ammonium polyacryldimethyltauramide) is heated to about 75° C. The ammonium polyacryldimethyltauramide is incorporated into the rest of phase B, which is stirred until a homogeneous gel is obtained.

Phase A is also heated to about 75° C. and an emulsion is prepared by incorporating this phase A into phase B.

When this emulsion has a temperature of 40-45° C., phase C is incorporated therein and, where appropriate (Example 2B), phase D. The emulsion is stirred and stirring is continued until cooling is complete.

EXAMPLE 1A
Oil-in-water emulsion (comparative).

| | | Concen- |
|---|---|---|
| | | tration |
| Phase | Name | (mass %) |
| A | Mixture of glyceryl stearate and of polyethylene | 2.00 |
| | glycol-100 stearate (Arlacel 165FL from Uniqema) | |
| | Oxyethylenated (25 OE) oxypropylenated (25 OP) | 1.50 |
| | mixture of dimyristyl tartrate, cetearyl alcohol | |
| | and lauryl alcohol (Cosmacol PSE from Sasol) | |
| | Cyclohexasiloxane | 10.00 |
| | Stearyl alcohol | 1.00 |
| B | Water | qs 100 |
| | Preserving agents | 0.75 |
| | Pentasodium salt of ethylenediamine- | 0.05 |
| | tetramethylenephosphonate | |
| | Ammonium polyacryldimethyltauramide | 0.40 |
| | (Hostacerin AMPS from Clariant) | |
| | Xanthan gum (Rhodicare S from Rhodia) | 0.20 |
| C | Aqueous dispersion of colloidal silica (Cosmo S40 | 17.10 |
| | from Catalysts & Chemicals) | |

EXAMPLE 1B
Anti-wrinkle cosmetic composition - oil-in-water emulsion

| | | Concen- |
|---|---|---|
| | | tration |
| Phase | Name | (mass %) |
| A | Mixture of glyceryl stearate and of polyethylene | 2.00 |
| | glycol-100 stearate (Arlacel 165FL from Uniqema) | |
| | Oxyethylenated (25 OE) oxypropylenated (25 OP) | 1.50 |
| | mixture of dimyristyl tartrate, cetearyl alcohol | |
| | and lauryl alcohol (Cosmacol PSE from Sasol) | |
| | Cyclohexasiloxane | 10.00 |
| | Stearyl alcohol | 1.00 |
| B | Water | qs 100 |
| | Preserving agents | 0.75 |
| | Pentasodium salt of ethylenediamine- | 0.05 |
| | tetramethylenephosphonate | |
| | Ammonium polyacryldimethyltauramide | 0.40 |
| | (Hostacerin AMPS from Clariant) | |
| | Xanthan gum (Rhodicare S from Rhodia) | 0.20 |
| C | Aqueous dispersion of colloidal silica (Cosmo S40 | 17.10 |
| | from Catalysts & Chemicals) | |
| D | Polyamide fibres (Nylon 66 - 12185 polyamide pulp | 2.50 |
| | from Paul Bonte) | |

### Example 2

Demonstration of the Improvement of the Remanence Properties of the Formulae According to the Invention

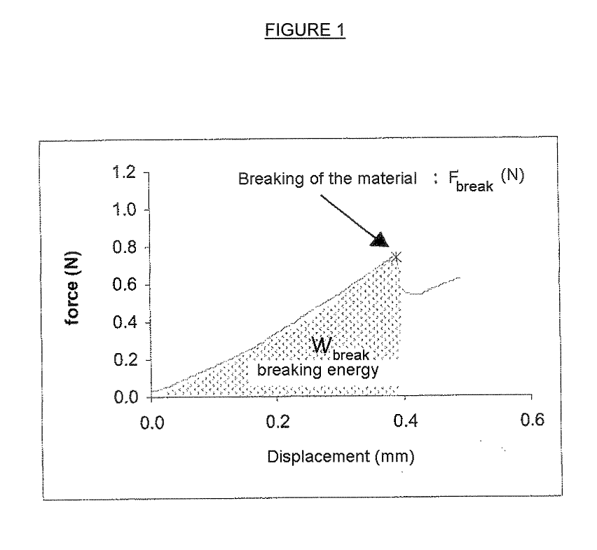

The test consisted in subjecting a material (in the present case the anti-wrinkle creams of Examples 1A and 1 B) deposited on the surface of a flexible and deformable foam, to compression up to the breaking point. The use of this foam support makes it possible to impose a large deformation on the material deposited on the surface, and thus to quantify its breaking strength. The mechanical compression stress was exerted using a cylindrical punch 1 mm in diameter; the travelling speed of the punch was 0.1 mm/s. The test was performed using a TA-XT2i texture analyser sold by the company Stable Micro System. A curve of the force F (in N) as a function of the displacement d (in mm) was thus obtained, from which it was possible to determine the breaking point of the material. FIG. 1 shows an example of a curve of force as a function of the displacement.

The parameter Wbreak (breaking energy in J/m²) is adopted to quantify the breaking strength of the material. The parameter corresponding to the area under the curve F=f(d)/area of the punch.

The substrate consisted of a neoprene foam 13 mm thick. The material (anti-wrinkle composition) was deposited onto this substrate so as to obtain, after drying for 24 hours, a film 15 to 30 μm thick. The deposits were produced using a film spreader that deposits 650 μm wet.

The results obtained are as follows:

| Material | W_{break} (J/m²) |
|---|---|
| Example 1A (comparative) | 15 ± 4 |
| Example 1B | 171 ± 246 |

It was found that the polyamide fibres used in the composition of Example 1B have a reinforcing role, this reinforcing role being illustrated by an increase in the breaking energy. This results in a more remanent tensioning effect of the composition of Example 1 B. Specifically, if the tensioning effect is associated with the formation of a rigid deposit, it is understood that the durability of this effect is associated with the mechanical strength of this deposit.

It was moreover confirmed on a panel of 6 women that the composition of Example 1B has a satisfactory tensioning effect.

The above written description of the invention provides a manner and process of making and using it such that any person skilled in this art is enabled to make and use the same, this enablement being provided in particular for the subject matter of the appended claims, which make up a part of the original description and including a composition comprising, in a physiologically acceptable medium, at least one aqueous dispersion of colloidal particles of at least one mineral filler, the particles having a number-average diameter of between 0.1 and 100 nm, and at least one type of organic fibres.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

The invention method and composition is preferably used by subjects desirous of the benefits noted herein, subjects “in need of” these benefits. Such subjects are typically suffering from signs of ageing of the skin generally, from age-related hollowing of the face and/or cheeks, or from age-related changes to the contour of the eyes, such as by self diagnosis or cosmetician or medical diagnosis, and are using the invention to smooth out human facial and/or bodily skin and/or for reducing or effacing the signs of ageing of the skin, in particular for reducing or effacing skin wrinkles and/or fine lines

Naturally, one using the invention as disclosed will use an amount of the invention composition effective to provide at least one of the benefits noted herein. Such amount is inclusive of an amount of the compositions described herein at the disclosed concentrations of active ingredients sufficient to cover the area of the skin being treated in a single application, and of course includes that amount applied upon repeated application, for example on a daily basis over a course of days, weeks, etc. In a preferred embodiment the invention process includes multiple applications of the invention composition to the area(s) of skin in need of attention.

1. A composition comprising, in a physiologically acceptable medium, at least:
one aqueous dispersion of colloidal particles of at least one mineral filler, the particles having a number-average diameter of between 0.1 and 100 nm, and
at least one type of organic fibres.

2. The composition according to claim 1, wherein the mineral filler is chosen from:
silica, cerium oxide, zirconium oxide, alumina, silicon carbonate, barium sulfate, calcium sulfate, zinc oxide, titanium dioxide, platinum and mixtures thereof.

3. The composition according to claim 1, wherein the mineral filler is a mixed oxide.

4. The composition according to claim 3, wherein the mineral filler is a silica-alumina composite filler.

5. The composition according to claim 4, wherein the surface area of the particles that is covered with aluminium is 4%-6%.

6. The composition according to claim 4, wherein the particles have a zeta potential of less than −25 mV, at pH 7 and at 25° C.

7. The composition according to claim 6, wherein the particles have a number-average diameter of 3 -30 nm.

8. The composition according to claim 1, wherein the colloidal particles of mineral filler are present in a content of 0.01%-15% by weight relative to the total weight of the composition.

9. The composition according to claim 1, wherein the fibres have a yarn count of 0.15 -30 denier.

10. The composition according to claim 1, wherein the fibres have a shape factor of 3.5-2500.

11. The composition according to claim 1, wherein the fibres have a length of 1 μm-10 mm.

12. The composition according to claim 1, wherein the fibres have a cross section that is within a circle of diameter of 1 nm-100 μm.

13. The composition according to claim 1, wherein the fibres are chosen from silk fibres, cotton fibres, wool fibres, flax fibres, cellulose fibres, polyamide fibres, modified cellulose fibres, poly-p-phenyleneterephthalamide fibres, acrylic fibres, especially polymethyl methacrylate or poly(2-hydroxyethyl methacrylate) fibres, polyolefin fibres and especially polyethylene or polypropylene fibres, aramid fibres, polytetrafluoroethylene fibres, insoluble collagen fibres, polyester fibres, polyvinyl chloride or polyvinylidene chloride fibres, polyvinyl alcohol fibres, polyacrylonitrile fibres, chitosan fibres, polyurethane fibres, polyethylene phthalate fibres, fibres of a copolymer of lactic acid and glycolic acid, terephthalate polyester fibres, fibres formed from a mixture of polymers, and mixtures thereof.

14. The composition according to claim 13, wherein the fibres are chosen from polyamide fibres, poly-p-phenyleneterephthalamide fibres, cotton fibres, and mixtures thereof.

15. The composition according to claim 1, wherein the fibres are treated and/or coated fibres.

16. The composition according to claim 1, wherein the fibres are present in an amount ranging from 0.1% to 50% by weight relative to the total weight of the composition.

17. The composition according to claim 1, wherein it is in the form of a serum, a lotion, a direct emulsion (O/W), inverse emulsion (W/O), multiple emulsion (O/W/O and W/O/W), a stick or a compact product.

18. The composition according to claim 1, wherein the composition is an anti-wrinkle composition.

19. The composition according to claim 1, wherein the composition is a foundation.

20. A process for treating the skin, comprising applying the composition of claim 1 to the skin.
